(19) United States
(12) Patent Application Publication
(10) Pub. No.: US 2005/0084883 A1
(43) Pub. Date: Apr. 21, 2005

(54) Method of diagnosis and treatment of pancreatic endocrine neoplasms based on differential gene expression analysis

(75) Inventors: Anirban Maitra, Kensington, MD (US); Charles J. Yeo, Cockeysville, MD (US); Donna E. Hansel, Baltimore, MD (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: The Johns Hopkins University School of Medicine, Baltimore, MD (US)

(21) Appl. No.: 10/925,864

(22) Filed: Aug. 25, 2004

Related U.S. Application Data
(60) Provisional application No. 60/497,606, filed on Aug. 25, 2003.

(51) Int. Cl. C12Q 1/68

(52) U.S. Cl. 435/6

(57) ABSTRACT

Methods for diagnosing pancreatic endocrine neoplasms are provided. Methods include obtaining a nucleic acid sample from the subject; contacting nucleic acids of the sample with an array comprising a plurality of DNAs under conditions to form one or more hybridization complexes; detecting the hybridization complexes; and comparing the levels of the hybridization complexes detected with the level of hybridization complexes detected in a non-diseased sample, wherein an altered level of hybridization complexes detected compared with the level of hybridization complexes of a non-diseased sample correlates with the presence of PEN in the subject. Nearly 200 differentially expressed genes identified in well-differentiated PENs versus enriched normal islet cells are provided, and a subset of these genes was validated at the protein level using PEN tissue microarrays.

### CROSS REFERENCE TO RELATED APPLICATION(S)

This application claims the benefit of priority under 35 U.S.C. § 119(e) of U.S. Ser. No. 60/497,606, filed Aug. 25, 2003, the entire contents of which are incorporated herein by reference.

### BACKGROUND OF THE INVENTION

1. Field of the Invention

The invention relates generally to pancreatic neoplasms and more specifically to differential gene expression in pancreatic endocrine neoplasms (PENs) as compared with normal islet cells and use of this information for diagnostic and therapeutic purposes.

2. Background Information

Within the past decade, several technologies have made it possible to monitor the expression level of a large number of transcripts within a cell at any one time (see, e.g., Schena et al., 1995, Quantitative monitoring of gene expression patterns with a complementary DNA micro-array, Science 270:467-470; Lockhart et al., 1996, Expression monitoring by hybridization to high-density oligonucleotide arrays, Nature Biotechnology 14:1675-1680; Blanchard et al., 1996, Sequence to array: Probing the genome's secrets, Nature Biotechnology 14, 1649; 1996, U.S. Pat. No. 5,569,588, issued Oct. 29, 1996 to Ashby et al. entitled “Methods for Drug Screening”). In organisms for which the complete genome is known, it is possible to analyze the transcripts of all genes within the cell. With other organisms, such as human, for which there is an increasing knowledge of the genome, it is possible to simultaneously monitor large numbers of the genes within the cell.

Early applications of this technology have involved identification of genes which are up regulated or down regulated in various diseased states. Additional uses for transcript arrays have included the analyses of members of signaling pathways, and the identification of targets for various drugs.

DNA-based arrays can provide a simple way to explore the expression of a single polymorphic gene or a large number of genes. When the expression of a single gene is explored, DNA-based arrays are employed to detect the expression of specific gene variants. For example, a p53 tumor suppressor gene array is used to determine whether individuals are carrying mutations that predispose them to cancer. The array has over 50,000 DNA probes to analyze more than 400 distinct mutations of p53. A cytochrome p450 gene array is useful to determine whether individuals have one of a number of specific mutations that could result in increased drug metabolism, drug resistance or drug toxicity.

DNA-based array technology is especially relevant for the rapid screening of expression of a large number of genes. There is a growing awareness that gene expression is affected in a global fashion. A genetic predisposition, disease or therapeutic treatment may affect, directly or indirectly, the expression of a large number of genes. In some cases the interactions may be expected, such as where the genes are part of the same signaling pathway. In other cases, such as when the genes participate in separate signaling pathways, the interactions may be totally unexpected. Therefore, DNA-based arrays can be used to investigate how genetic predisposition, disease, or therapeutic treatment affects the expression of a large number of genes.
cDNA-based arrays have been used in discovery and analysis of inflammatory disease related genes (Heller et al. (1997) Proc. Natl. Acad. Sci USA 94: 2150-2155). A first type of array was employed to characterize the expression patterns of a class of 96 genes coding for polypeptides known to be involved in rheumatoid arthritis. This array contained preselected probes for the 96 genes. A second type of array was used to investigate gene expression patterns characteristic of blood cells. This array contained probes for 1,000 human genes randomly selected from a human blood cell cDNA library.

Despite several advances in our basic understanding and in the clinical management of pancreatic cancer, virtually all patients who will be diagnosed with pancreatic cancer will die from the disease. The high mortality of pancreatic cancer is predominantly because of diagnosis at an advanced stage of disease and lack of effective treatments. New tumor markers of pancreatic cancer are needed to allow increased ability to diagnose the disease at an early stage.

Well-differentiated pancreatic endocrine neoplasms (PENs), commonly referred to as islet cell tumors, are a unique group of malignancies often characterized by a clinical neuroendocrine syndrome attributable to the selective overproduction and humoral circulation of pancreas-specific hormones^{1,2} Approximately 2,000 new cases of well-differentiated PENs are diagnosed each year in the United States, and of these cases, 60-70% are associated with a clinical syndrome resulting from the secretion of a single functional hormone³. The remaining one-third of PENs secrete no clinically detectable biologically active hormones and most often present as space occupying lesions causing obstructive jaundice, upper gastrointestinal luminal obstruction, bleeding or abdominal pain. While the majority of well-differentiated PENs follow an indolent course, a substantial proportion of nonfunctional tumors are defined by aggressive biology resulting in early locoregional invasion of lymph node basins and adjacent organs, as well as metastases to the liver and beyond³. Several histopathologic parameters, including tumor size, pleomorphism, angiolymphatic invasion, mitotic index, Ki-67 labeling index, and ploidy have been used for predicting long-term outcome following surgical resection However, apart from locoregional invasion and distant metastasis, there are no consistent determinants of malignant behavior for these neoplasms.

In contrast to the more common exocrine ductal adenocarcinomas, little is known about the molecular abnormalities or genotype-phenotype correlations that underlie PENs⁸. This is likely a reflection of the small numbers of cases that are accessible for molecular studies. As a result, diagnostic and therapeutic approaches for these neoplasms have seen few advances in the last decade. The power of global expression microarrays have previously been show to identify a variety of novel tumor markers in exocrine pancreatic ductal adenocarcinomas, with immediate translational potential for patient care^{9,10}.

### SUMMARY OF THE INVENTION

Pancreatic endocrine neoplasms (PENs) are rare, mostly well-differentiated endocrine neoplasms, whose biology has been poorly characterized. Global expression microarrays provide an excellent resource for documenting abnormal pathways that may impact on tumorigenesis and disease progression in these neoplasms. The studies shown herein document aberrantly activated pathways that may impact on tumorigenesis, and identify potential cellular targets in these uncommon tumors. Nearly 200 differentially expressed genes identified in well-differentiated PENs versus enriched normal islet cells are provided, and a subset of these genes was validated at the protein level using PEN tissue microarrays. The overwhelming majority of differentially expressed genes identified herein have not been previously described in PENs and may serve as potential therapeutic targets and novel tumor markers for this disease.

In one embodiment, the invention provides a method for diagnosing pancreatic endocrine neoplasm (PEN) in a subject including obtaining a nucleic acid sample from the subject; contacting nucleic acids of the sample with an array comprising a plurality of DNAs under conditions to form one or more hybridization complexes; detecting the hybridization complexes; and comparing the levels of the hybridization complexes detected above with the level of hybridization complexes detected in a non-diseased sample, wherein an altered level of hybridization complexes detected above compared with the level of hybridization complexes of a non-diseased sample correlates with the presence of PEN in the subject. Typically, the DNAs are immobilized on a substrate and may be in the form of a microarray. In one aspect, at least one nucleic acid sequence is selected from Table 2 or Table 3 and combinations thereof. For example, a differentially expressed nucleic acid may include a nucleic acid sequence encoding a putative oncogene, growth factor, cell adhesion and migration molecule, endothelial element, cell cycle checkpoint protein, metastasis suppressor gene, transcription factor, or a combination thereof. Illustrative genes that are differentially expressed include but are not limited to MLLT10/AF10, IGFBP3, fibronectin, MUC18, p21/Cip1, GADD45, NME3, junD, or a combination thereof.

In another embodiment, the invention provides a method for predicting the likelihood of metastases from a primary pancreatic endocrine neoplasm (PEN) in a subject. The method includes obtaining a nucleic acid sample from the subject; contacting nucleic acids of the sample with a nucleic acid sequence that is complementary to IGFBP3 under conditions that allow formation of hybridization complexes; detecting the hybridization complexes; and comparing the levels of the hybridization complexes detected above with the level of hybridization complexes detected in a non-diseased sample, wherein an altered level of hybridization complexes detected above compared with the level of hybridization complexes of a non-diseased sample correlates with the likelihood of developing metastases in the subject.

In another embodiment, the invention provides a method for predicting the likelihood of metastases from a primary pancreatic endocrine neoplasm (PEN) in a subject including obtaining a nucleic acid sample from the subject; contacting nucleic acids of the sample with a nucleic acid sequence that is complementary to fibronectin under conditions that allow formation of hybridization complexes; detecting the hybridization complexes; and comparing the levels of the hybridization complexes detected above with the level of hybridization complexes detected in a non-diseased sample, wherein an altered level of hybridization complexes detected above compared with the level of hybridization complexes of a non-diseased sample correlates with the likelihood of developing metastases in the subject.

In another embodiment, the invention provides a method for diagnosing pancreatic endocrine neoplasm (PEN) in a subject including obtaining a nucleic acid sample from the subject; contacting nucleic acids of the sample with a nucleic acid sequence that is complementary to CD99 under conditions that allow formation of hybridization complexes; detecting the hybridization complexes; and comparing the levels of the hybridization complexes detected above with the level of hybridization complexes detected in a non-diseased sample, wherein an altered level of hybridization complexes detected above compared with the level of hybridization complexes of a non-diseased sample correlates with the presence of PEN in the subject.

The invention also provides a method for diagnosing pancreatic endocrine neoplasm (PEN) in a subject including obtaining a nucleic acid sample from the subject; contacting nucleic acids of the sample with a nucleic acid sequence that is complementary to p21 under conditions that allow formation of hybridization complexes; detecting the hybridization complexes; and comparing the levels of the hybridization complexes detected above with the level of hybridization complexes detected in a non-diseased sample, wherein an altered level of hybridization complexes detected above compared with the level of hybridization complexes of a non-diseased sample correlates with the presence of PEN in the subject.

In another embodiment, the invention provides a method for monitoring the course of treatment in a subject having a pancreatic endocrine neoplasm (PEN) including obtaining a nucleic acid sample from the subject; contacting nucleic acid of the sample with an array comprising a plurality of DNAs under conditions to form one or more hybridization complexes; detecting said hybridization complexes; and comparing the levels of the hybridization complexes detected above with the level of hybridization complexes detected in the subject prior to and during the course of treatment, wherein an altered level of hybridization complexes detected prior to and during treatment correlates with the effectiveness of treatment in the subject.

In one embodiment, the invention provides a method of identifying a pattern of polynucleotide expression for PENs versus normal human islet cells. The method includes hybridizing nucleic acid obtained from cells of PENs or cells suspected of being PENs or being at risk for developing into PENs with a gene expression microarray to form a pattern of expression and comparing the pattern with a pattern obtained from hybridizing nucleic acid obtained from normal human islet cells. A difference in the patterns is indicative of expression of genes, whether up-regulated or down-regulated, specific for tumorigenesis or disease progression.

In one embodiment, the invention provides a method of identifying a pattern of polynucleotide expression for identification of a compound that affects tumor or disease progression of a PEN. The method includes hybridizing nucleic acid obtained from cells of PENs or cells suspected of being PENs or being at risk for developing into PENs with at least one gene or a gene expression microarray to form a pattern of expression and comparing the pattern with a pattern obtained from hybridizing nucleic acid obtained from cells of PENs or cells suspected of being PENs or being at risk for developing into PENs following treatment with an agent or compound for treating PEN cells. A difference in the patterns is indicative of an effect of the agent or compound on expression of genes, whether up-regulated or down-regulated, and thus an affect on tumorigenesis or disease progression.

In one aspect, when fibronectin or IGFBP3 are detected, expression during the course of treatment is decreased as compared to prior to treatment. In another aspect, when CD99 or p21 are detected, expression during the course of treatment is increased as compared to prior to treatment.

The invention also provides a method for identifying a cell that exhibits or is predisposed to exhibiting unregulated growth, comprising detecting, in a test cell, at least one gene expressed differentially as compared to a normal islet cell, wherein the at least one gene is set forth in Table 2 or Table 3, or a combination thereof, thereby identifying the test cell as a cell that exhibits or is predisposed to exhibiting unregulated growth.

In yet another embodiment, the invention provides a method of treating a pancreatic endocrine neoplasm in a subject including contacting islet cells of the subject with a compound or agent that affects expression of one or more genes associated with the neoplasm, wherein the one or more gene is selected from a gene set forth in Table 2 or 3 or a combination thereof. The affect of the compound or agent on expression may be upregulation or it may be downregulation of expression.

In yet another embodiment, the invention provides a including a tissue microarray generated from tissue obtained from a subject having a PEN. Optionally, the microarray contains tissue from lymph node metastases and/or hepatic metastases without the primary tumor. The kit may also include reagents for immunohistochemical labeling of tissue, such as avidin, biotin and peroxidase as well as antibodies specific for proteins identified as associated with PENs, such as fibronectin or IGFBP3, for example.

### DETAILED DESCRIPTION OF THE INVENTION

The present invention is based on studes of gene expression in well-differentiated PENs; these studies document aberrantly activated pathways that may impact on tumorigenesis, and identify potential cellular targets in these uncommon tumors. Nearly 200 differentially expressed genes in well-differentiated PENs versus enriched normal islet cells are shown and validated using a subset of these genes at the protein level using PEN tissue microarrays. The overwhelming majority of differentially expressed genes identified have not been previously described in PENs and may serve as potential therapeutic targets and novel tumor markers for this disease.

General methodology included the following. RNA was extracted from eight well-differentiated PENs and three highly enriched pancreatic islet cell samples (80-90% purity), and examined using the Affymetrix U133A oligonucleotide microarrays, containing over 22,000 known human transcripts. Microarray data were normalized using dCHIP (www.dCHIP.org) for identification of differentially expressed genes. PEN tissue microarrays were constructed from 53 archival PENs for immunohistochemical validation of microarray data. 66 transcripts were overexpressed 3-fold or higher in PENs compared to normal islet cells, including putative oncogenes (MLLT10/AF10), growth factors (insulin like-growth factor binding protein 3 [IGFBP3]), cell adhesion and migration molecules (fibronectin), and endothelial elements (MUC18/MelCAM, CD31). 119 transcripts were underexpressed 3-fold or less in PENs compared to normal islet cells, including cell cycle checkpoint proteins (p21/Cip1), the MIC2 (CD99) cell surface glycoprotein, genes involved in DNA damage repair and genomic stability (O-6-methylguanine-DNA methyltransferase, GADD45), putative metastasis suppressor genes (NME3), andjunD, a MEN1-regulated transcription factor. Using PEN tissue microarrays, differential upregulation of IGFBP3 (70%) and fibronectin (22%), and differential downregulation of p21 (46%) and MIC2 (CD99) (91%) in PENs versus normal pancreatic islets was confirmed. IGFBP3 overexpression was significantly more common in metastatic (93%) versus primary PEN lesions (60%), P=0.022. Fibronectin overexpression demonstrated a trend towards significance in lymphatic PEN metastases (55%), compared to primary PEN lesions (24%), P=0.14. Global expression analysis provides an insight into tumorigenic pathways in PENs, and may identify potential prognostic and therapeutic markers for these uncommon neoplasms.

The term “microarray” refers to an ordered arrangement of hybridizable array elements. The array elements are arranged so that there are preferably at least one or more different array elements, more preferably at least 100 array elements, and most preferably at least 1,000 array elements, on a 1 cm2 substrate surface. The maximum number of array elements is unlimited, but is at least 100,000 array elements. Furthermore, the hybridization signal from each of the array elements is individually distinguishable. In a preferred embodiment, the array elements comprise polynucleotide probes. The term “microarray,” as used herein, also refers to an arrangement of distinct polynucleotides or oligonucleotides on a substrate, such as paper, nylon or any other type of membrane, filter, chip, glass slide, or any other suitable solid support.

A cell or test cell, is a cell exhibiting, or predisposed to exhibiting unregulated growth, can be a neoplastic cell, for example, a premalignant cell or a malignant cell (i.e., a cancer cell). As such, the cell can be a cell known or suspected of being a carcinoma cell or the like. A cell proliferative disorder as described herein may be a neoplasm. Such neoplasms are either benign or malignant. The term “neoplasm” refers to a new, abnormal growth of cells or a growth of abnormal cells that reproduce faster than normal. A neoplasm creates an unstructured mass (a tumor) which can be either benign or malignant. The term “benign” refers to a tumor that is noncancerous, e.g. its cells do not invade surrounding tissues or metastasize to distant sites. The term “malignant” refers to a tumor that is metastastic, invades contiguous tissue or no longer under normal cellular growth control. In one embodiment, the cell exhibiting or predisposed to exhibiting unregulated growth, or suspected of being such a cell is a cancer cell, and more specifically, a pancreatic endocrine neoplastic cell or an islet cell.

A “polynucleotide” refers to a chain of nucleotides. Preferably, the chain has from about 100 to 10,000 nucleotides, more preferably from about 150 to 3,500 nucleotides. The term “probe” refers to a polynucleotide sequence capable of hybridizing with a target sequence to form a polynucleotide probe/target complex. A “target polynucleotide” refers to a chain of nucleotides to which a polynucleotide probe can hybridize by base pairing. In some instances, the sequences will be complementary (no mismatches). In other instances, there may be a 10%, mismatch.

A “plurality” refers preferably to a group of at least one or more members, more preferably to a group of at least about 100, and even more preferably to a group of at least about 1,000, members. The maximum number of members is unlimited, but is at least about 100,000 members.

A “portion” means a stretch of at least about 100 consecutive nucleotides. A “portion” can also mean a stretch of at least 100 consecutive nucleotides that contains one or more deletions, insertions or substitutions. A “portion” can also mean the whole coding sequence of a gene. Preferred portions are those that lack secondary structure as identified by using computer software programs such as OLIGO 4.06 Primer Analysis is Software, (National Biosciences, Plymouth, Minn.) LASERGENE software (DNASTAR, Madison Wis., MACDASIS software (Hitachi Software Engineering Co., Ltd. South San Francisco, Calif.) and the like.

The term “gene” or “genes” refers to the partial or complete coding sequence of a gene. The phrase “associated with PEN” refers to genes that are known to regulate cellular proliferation or unknown genes that are differentially or abundantly expressed in PEN as compared with “normal” tissue or tissues not having the disease, and include those listed in the Sequence Listing and in Table 1.

The phrase “differentially expressed gene” refers to a gene whose abundance in a target transcript profile is preferably at least about 1.5 times higher or lower, more preferably about 2 times higher or lower, or 3 times higher or lower than that in a normal cell transcript profile. The phrase also refers to genes that are not detectable in the normal cell transcript profile but are preferably at levels of at least about 2 copies per cell, more preferably at least about 3 copies per cell, in the target transcript profile. “Abundantly expressed gene” refers to a gene which represents preferably at least about 0.01% of the transcripts in a transcript profile.

As used herein, the profile of transcripts which reflect gene expression in a particular tissue, at a particular time, is defined as a “transcript profile”. Such profiles can be generated by naming, matching, and counting all copies of related clone inserts and arranging them in order of abundance. A “target transcript profile” refers to a profile derived from a biological sample that contains transcripts of interest along side transcripts which are not of interest.

“Antibodies” as used herein includes polyclonal and monoclonal antibodies, chimeric, single chain, and humanized antibodies, as well as Fab fragments, including the products of an Fab or other immunoglobulin expression library.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

Reference herein to “nucleic acid molecules corresponding to RNA” of a cell means RNA such as mRNA or polyA+ RNA, cDNA generated using RNA from the cell as a template, or cRNA generated using RNA or cDNA as a template. For practicing a method of the invention, the nucleic acid molecules corresponding to RNA of a cell generally are detectably labeled, for example, with a radioisotope, a paramagnetic isotope, a luminescent compound, a chemiluminescent compound, a fluorescent compound, a metal chelate, an enzyme, a substrate for an enzyme, a receptor, or a ligand for a receptor; or are capable of being detected, for example, using a detectably labeled probe, such that hybridization of the nucleic acid molecules to nucleotide sequences of the array can be detected. Thus, the nucleic acid molecules corresponding to RNA that are contacted with the nucleotide sequences of the array can be DNA or RNA, including, for example, cDNA, cRNA, mRNA, or any other nucleic acid molecules representative of RNA expressed in a cell.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques which are well known in the art.

The terms “complementary” or “complementarity,” as used herein, refer to the natural binding of polynucleotides under permissive salt and temperature conditions by base pairing. For example, the sequence “A-G-T” binds to the complementary sequence “T-C-A.” Complementarity between two single-stranded molecules may be “partial,” such that only some of the nucleic acids bind, or it may be “complete,” such that total complementarity exists between the single stranded molecules. The degree of complementarity between nucleic acid strands has significant effects on the efficiency and strength of the hybridization between the nucleic acid strands. This is of particular importance in amplification reactions, which depend upon binding between nucleic acids strands, and in the design and use of peptide nucleic acid (PNA) molecules. A “composition comprising a given polynucleotide sequence” or a “composition comprising a given amino acid sequence,” as these terms are used herein, refer broadly to any composition containing the given polynucleotide or amino acid sequence. The composition may comprise a dry formulation, an aqueous solution, or a sterile composition. Probes may be stored in freeze-dried form and may be associated with a stabilizing agent such as a carbohydrate. In hybridizations, the probe may be deployed in an aqueous solution containing salts (e.g., NaCl), detergents (e.g., SDS), and other components (e.g., Denhardt's solution, dry milk, salmon sperm DNA, etc.).

As used herein, the term “correlates with expression of a polynucleotide ” (or gene) indicates that the detection of the presence of nucleic acids, the same or related to a nucleic acid sequence encoding particular proteins as determined by hybridization with a microarray is indicative of the presence of nucleic acids encoding a protein associated with PENs or tumorigenesis or disease progression in a sample.

The term “homology,” as used herein, refers to a degree of complementarity. There may be partial homology or complete homology. The word “identity” may substitute for the word “homology.” A partially complementary sequence that at least partially inhibits an identical sequence from hybridizing to a target nucleic acid is referred to as “substantially homologous.” The inhibition of hybridization of the completely complementary sequence to the target sequence may be examined using a hybridization assay (e.g., a microarray, Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

The phrases “percent identity” or “% identity” refer to the percentage of sequence similarity found in a comparison of two or more amino acid or nucleic acid sequences. Percent identity can be determined electronically, e.g., by using the MegAlign program (DNASTAR, Inc., Madison Wis.). This program can create alignments between two or more sequences according to different methods, e.g., the clustal method. (Higgins, D. G. and P. M. Sharp (1988) Gene 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be counted or calculated by other methods known in the art, such as the Jotun Hein method. (See, e.g., Hein, J. (1990) Methods Enzymol. 183:626-645.) Identity between sequences can also be determined by other methods known in the art, e.g., by varying hybridization conditions.

“Hybridization,” as the term is used herein, refers to any process by which a strand of nucleic acid binds with a complementary strand through base pairing. As used herein, the term “hybridization complex” as used herein, refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The term “modulate,” as it appears herein, refers to a change in expression of a gene. For example, modulation may cause an increase or a decrease in expression, protein activity, binding characteristics, or any other biological, functional, or immunological property.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material. In this context, “fragments” refers to those nucleic acid sequences which are greater than about 60 nucleotides in length, and most preferably are at least about 100 nucleotides, at least about 1000 nucleotides, or at least about 10,000 nucleotides in length.

The term “oligonucleotide,” as used herein, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay or microarray. As used herein, the term “oligonucleotide” is substantially equivalent to the terms “amplimer,” “primer,” “oligomer,” and “probe,” as these terms are commonly defined in the art.

The term “sample,” as used herein, is used in its broadest sense. A biological sample suspected of containing nucleic acids associated with tumorigenesis or disease progression or normal human islet cells, or fragments thereof, may comprise a bodily fluid; an extract from a cell, chromosome, organelle, or membrane isolated from a cell; a cell; genomic DNA, RNA, or cDNA, in solution or bound to a solid support; a tissue; a tissue print; and the like. Samples may include the primary tumor or lymph nodes, for example.

As used herein, the term “stringent conditions” refers to conditions which permit hybridization between polynucleotide sequences and the claimed polynucleotide sequences. Suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature.

For example, hybridization under high stringency conditions could occur in about 50% formamide at about 37 degrees C. to 42 degrees C. Hybridization could occur under reduced stringency conditions in about 35% to 25% formamide at about 30 degrees C. to 35 degrees C. In particular, hybridization could occur under high stringency conditions at 42 degrees C. in 50% formamide, 5× SSPE, 0.3% SDS, and 200 μg/ml sheared and denatured salmon sperm DNA. Hybridization could occur under reduced stringency conditions as described above, but in 35% formamide at a reduced temperature of 35 degrees C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

The term “substantially purified,” as used herein, refers to nucleic acid or amino acid sequences that are removed from their natural environment and are isolated or separated, and are at least about 60% free, preferably about 75% free, and most preferably about 90% free from other components with which they are naturally associated.

In one embodiment, the invention provides a method for diagnosing pancreatic endocrine neoplasm (PEN) in a subject including obtaining a nucleic acid sample from the subject; contacting nucleic acids of the sample with an array comprising a plurality of DNAs under conditions to form one or more hybridization complexes; detecting the hybridization complexes; and comparing the levels of the hybridization complexes detected above with the level of hybridization complexes detected in a non-diseased sample, wherein an altered level of hybridization complexes detected above compared with the level of hybridization complexes of a non-diseased sample correlates with the presence of PEN in the subject. Typically, the DNAs are immobilized on a substrate and may be in the form of a microarray. In one aspect, the at least one nucleic acid sequence is selected from Table 2 or Table 3 and combinations thereof. For example, a differentially expressed nucleic acid may include a nucleic acid sequence encoding a putative oncogene, growth factor, cell adhesion and migration molecule, endothelial element, cell cycle checkpoint protein, metastasis suppressor gene, transcription factor, or a combination thereof. Illustrative genes that are differentially expressed include but are not limited to MLLT10/AF10, IGFBP3, fibronectin, MUC18, p21/Cip1, GADD45, NME3, junD, or a combination thereof. In one aspect, IGFBP3 is useful for as a predictor of metastases. In another aspect, fibronectin is useful as a therapeutic target in a subset of PEN lesions, particularly those with nodal metastases.

In another embodiment, the invention provides a method for predicting the likelihood of metastases from a primary pancreatic endocrine neoplasm (PEN) in a subject. The method includes obtaining a nucleic acid sample from the subject; contacting nucleic acids of the sample with a nucleic acid sequence that is complementary to IGFBP3 under conditions that allow formation of hybridization complexes; detecting the hybridization complexes; and comparing the levels of the hybridization complexes detected above with the level of hybridization complexes detected in a non-diseased sample, wherein an altered level of hybridization complexes detected above compared with the level of hybridization complexes of a non-diseased sample correlates with the likelihood of developing metastases in the subject. IGFBP3 is typically upregulated in PEN as compared with normal islet cells (e.g., human islet cells).

In another embodiment, the invention provides a method for predicting the likelihood of metastases from a primary pancreatic endocrine neoplasm (PEN) in a subject including obtaining a nucleic acid sample from the subject; contacting nucleic acids of the sample with a nucleic acid sequence that is complementary to fibronectin under conditions that allow formation of hybridization complexes; detecting the hybridization complexes; and comparing the levels of the hybridization complexes detected above with the level of hybridization complexes detected in a non-diseased sample, wherein an altered level of hybridization complexes detected above compared with the level of hybridization complexes of a non-diseased sample correlates with the likelihood of developing metastases in the subject. Fibronectin is typically upregulated in PEN as compared with normal islet cells (e.g., human islet cells).

In another embodiment, the invention provides a method for diagnosing pancreatic endocrine neoplasm (PEN) in a subject including obtaining a nucleic acid sample from the subject; contacting nucleic acids of the sample with a nucleic acid sequence that is complementary to CD99 under conditions that allow formation of hybridization complexes; detecting the hybridization complexes; and comparing the levels of the hybridization complexes detected above with the level of hybridization complexes detected in a non-diseased sample, wherein an altered level of hybridization complexes detected above compared with the level of hybridization complexes of a non-diseased sample correlates with the presence of PEN in the subject. CD99 is typically downregulated in PEN as compared with normal islet cells (e.g., human islet cells).

The invention also provides a method for diagnosing pancreatic endocrine neoplasm (PEN) in a subject including obtaining a nucleic acid sample from the subject; contacting nucleic acids of the sample with a nucleic acid sequence that is complementary to p21 under conditions that allow formation of hybridization complexes; detecting the hybridization complexes; and comparing the levels of the hybridization complexes detected above with the level of hybridization complexes detected in a non-diseased sample, wherein an altered level of hybridization complexes detected above compared with the level of hybridization complexes of a non-diseased sample correlates with the presence of PEN in the subject. p21 is typically downregulated in PEN as compared with normal islet cells (e.g., human islet cells).

The MIC2 gene locus is located in the pseudoautosomal (pairing) region of human X and Y chromosomes³⁵. The protein product of the MIC2 gene—CD99 or E2 antigen—is a 32-kD human T-cell surface glycoprotein involved in spontaneous rosette formation with erythrocytes²⁵. Intense membranous CD99 expression is considered a sine qua non of EWS-FL11 translocation positive primitive neuroectodermal tumors/Ewing sarcomas (PNET/ES)³⁶. CD99 reactivity has been increasingly recognized in a variety of other mesenchymal tumor types, including poorly differentiated synovial sarcomas and rhabdomyosarcomas, epithelial malignancies (gastric adenocarcinomas), and lymphoblastic lymphoma. In the pancreas, besides pancreatic PNET/ES³⁷, CD99 expression has been uncommonly reported in solid pseudopapillary tumors³⁸. Choi et al had previously reported absence of CD99 labeling in non-functioning islet cell tumors, but the pattern of expression in neoplastic versus non-neoplastic islet cells has not been previously described²⁹. In accordance with our microarray expression data, we found intense, diffuse CD99 expression in all benign islets, while 91% of PENs in our series lacked CD99 expression. Of note, Pelosi et al demonstrated CD99 labeling in ˜25% of neuroendocrine tumors arising in a variety of anatomic sites, and correlated CD99 expression with a low proliferation rate, as well as reduced rate of metastasis 39. The authors postulated that CD99 expression in neuroendocrine tumors might facilitate cell-cell adhesion, leading to decreased invasive properties. If this postulate were to hold true, then non-neoplastic islet cells would be expected to have the highest CD99 expression, as is indeed the case in our series. The mechanism of CD99 downregulation in PENs is a matter of speculation. Some studies have suggested a role for the Epstein Barr virus latent membrane protein (LMP 1) orchestrating downregulation of CD99 antigen via a NF kappaβ-mediated pathway in Reed-Sternberg cells^{40,41}; this is however unlikely to be the mechanism of action in PENs. We found no significant correlation between site of lesion and CD99 expression in our series—89% of primary tumors and 94% of hepatic and nodal metastases demonstrated lack of CD99 labeling. Only five primary PEN lesions on our TMAs demonstrated CD99 expression—4/5 (80%) were localized to the pancreas, while 1/5 (20%) was associated with nodal metastasis (also CD99 positive). These numbers are too small to draw a meaningful conclusion; nevertheless a larger series of cases may help clarify whether PENs with retained CD99 may represent a favorable prognostic subset.

IGFBP3 is a member of the family of insulin-like growth factors¹⁶. The insulin-like growth factors, their receptors, and their binding proteins play key roles in regulating cell proliferation and apoptosis. IGFBP3 is the major carrier protein for IGF1 and IGF2 in the circulation. IGFBP3 possesses both growth-inhibitory and -potentiating effects on cells that are independent of IGF action and are mediated through specific IGFBP3-binding proteins/receptors located at the cell membrane, cytosol, or nuclear compartments and in the extracellular matrix¹⁶. For example, in some cancer cells, IGFBP3 has pro-apoptotic activities both dependent on and independent of p53^{42,43}. On the contrary, elevated serum IGFBP3 may be a predictor for progression and recurrence of breast cancers^{17,44}. We have recently demonstrated upregulation of IGFBP3 in pancreatic ductal adenocarcinomas using the U133 microarray platform (unpublished data). The commonality of IGFBP3 upregulation in both exocrine and endocrine neoplasms of the pancreas suggests an important, possibly growth promoting role for this protein in pancreatic tumorigenesis. On immunohistochemistry, we found low to moderate “basal” IGFBP3 in normal islets, and overexpression in PENs was assessed by comparison with this “basal” level—a task greatly facilitated by the availability of adjacent non-neoplastic islets in 43/44 primary tumors on our PEN TMA. Using this criterion, 70% of PEN lesions demonstrated robust IGFBP3 overexpression. Although there was an excellent correlation between IGFBP3 labeling in matched primary and nodal metastases (9/9 or 100% of matched lesions demonstrated a similar pattern of IGFBP3 labeling), we found a significantly higher overall proportion of metastatic foci that labeled with IGFBP3 (15/16 or 93%) compared to primary tumors (24/40 or 60%) (P=0.022). In addition, within the subset of primary lesions only, 80% of primary PENs with metastasis demonstrated IGFBP3 overexpression, compared to 53% of localized PENs, although the difference did not reach statistical significance. Despite the relatively small numbers of cases in our series, we believe these immunohistochemical findings may be summarized as follows: (a) it appears that upregulation of IGFBP3 is an early event in PEN progression, usually occurring in the primary tumor itself, and (b) from a prognostic standpoint, upregulation of IGFBP3 may predict for a subset of PENs that have a propensity to metastasize.

Fibronectin is a 430,000-dalton dimeric glycoprotein that exists in 2 forms, termed cellular and plasma fibronectin. Cellular fibronectin is the major cell surface glycoprotein of many fibroblast cell lines, and serves as a ligand for the integrin family of cell adhesion receptors and regulates cytoskeletal organization^{19,20}. Several reports have demonstrated that secretion of this pro-migratory molecule may be a key event in cancer cells during the progression to a metastatic phenotype. For example, Bittner et al demonstrated overexpression of fibronectin in a series of highly metastatic uveal melanoma cell lines, consistent with an important role for focal contacts in modulating melanoma cell motility⁴⁵. Similarly, Clark et al reported that fibronectin was one of the three consistently upregulated genes (along with thymosinβ4 and RhoC) in pulmonary metastases arising from human and mouse melanoma cell lines⁴⁶. On the contrary, peptides that mimic the cell adhesive region of fibronectin were shown to inhibit metastasis, indicating that tumor cells must interact with molecules such as fibronectin to metastasize 47. In our series of PENs, we found expression of fibronectin in a small, but significant, minority of primary tumors (24%). Of interest however, lymphatic, but not hepatic, metastases of PENs demonstrated a higher proportion of cases with fibronectin expression—in 5/9 (55%) PENs, upregulation of fibronectin was restricted to the nodal metastasis only, while the primary lesion was negative (P=0.14). There was no significant difference in the proportion of primary PENs with associated metastasis expressing fibronectin (36%) versus localized PENs (19%), P=0.44. Thus, unlike IGFBP3, upregulation of fibronectin appears to be a late event in PEN progression, and in corroboration with previous reports, we also demonstrate a higher proportion of lymphatic metastases with fibronectin expression. The expression of fibronectin in the majority of lymphatic metastases of PENs also presents an opportunity to potentially use fibronectin-conjugated radionuclide- or immunotoxins for the treatment of refractory metastatic disease⁴⁸.

In another embodiment, the invention provides a method for monitoring the course of treatment in a subject having a pancreatic endocrine neoplasm (PEN) including obtaining a nucleic acid sample from the subject; contacting nucleic acid of the sample with an array comprising a plurality of DNAs under conditions to form one or more hybridization complexes; detecting said hybridization complexes; and comparing the levels of the hybridization complexes detected above with the level of hybridization complexes detected in the subject prior to and during the course of treatment, wherein an altered level of hybridization complexes detected prior to and during treatment correlates with the effectiveness of treatment in the subject. Thus, based on the findings of the present invention, prognostic as well as diagnostic tools can be developed for patients with PENs.

In one embodiment, the invention provides a method of identifying a pattern of polynucleotide expression for PENs versus normal islet cells. The method includes hybridizing nucleic acid obtained from cells of PENs or cells suspected of being PENs or being at risk for developing into PENs with a gene expression microarray to form a pattern of expression and comparing the pattern with a pattern obtained from hybridizing nucleic acid obtained from normal islet cells. A difference in the patterns is indicative of expression of genes, whether up-regulated or down-regulated, specific for tumorigenesis or disease progression. Preferably, the islet cells are human islet cells.

In one embodiment, the invention provides a method of identifying a pattern of polynucleotide expression for identification of a compound that affects tumor or disease progression of a PEN. The method includes hybridizing nucleic acid obtained from cells of PENs or cells suspected of being PENs or being at risk for developing into PENs with at least one gene or a gene expression microarray to form a pattern of expression and comparing the pattern with a pattern obtained from hybridizing nucleic acid obtained from cells of PENs or cells suspected of being PENs or being at risk for developing into PENs following treatment with an agent or compound for treating PEN cells. A difference in the patterns is indicative of an effect of the agent or compound on expression of genes, whether up-regulated or down-regulated, and thus an affect on tumorigenesis or disease progression.

Such compounds may include peptides, peptidomimetics, polypeptides, chemical compounds and biologic agents, for example.

In one aspect, when fibronectin or IGFBP3 are detected, expression during the course of treatment is decreased as compared to prior to treatment. In another aspect, when CD99 or p21 are detected, expression during the course of treatment is increased as compared to prior to treatment.

The invention also provides a method for identifying a cell that exhibits or is predisposed to exhibiting unregulated growth, comprising detecting, in a test cell, at least one gene expressed differentially as compared to a normal islet cell, wherein the at least one gene is set forth in Table 2 or Table 3, or a combination thereof, thereby identifying the test cell as a cell that exhibits or is predisposed to exhibiting unregulated growth.

In yet another embodiment, the invention provides a method of treating a pancreatic endocrine neoplasm in a subject including contacting islet cells of the subject with a compound or agent that affects expression of one or more genes associated with the neoplasm, wherein the one or more gene is selected from a gene set forth in Table 2 or 3 or a combination thereof. The affect of the compound or agent on expression may be upregulation or it may be downregulation of expression.

In yet another embodiment, the invention provides a kit including a tissue microarray generated from tissue obtained from a subject having a PEN. Optionally, the microarry contains tissue from lymph node metastases and/or hepatic metastases without the primary tumor. The kit may also include reagents for immunohistochemical labeling of tissue, such as avidin, biotin and peroxidase as well as antibodies specific for proteins identified as associated with PENs, such as fibronectin or IGFBP3, for example.

Another embodiment of the invention provides a method of determining a predisposition to a cellular proliferative disorder of pancreatic islet cells in a subject comprising determining the expression level of one or more nucleic acids isolated from the subject, wherein the nucleic acid is selected from at least one gene set forth in Table 2 or 3 or combinations thereof; and wherein level of expression of one or more nucleic acids as compared with the level of expression of the nucleic acid from a subject not having a predisposition to the cellular proliferative disorder of pancreatic islet cells or expression in normal islet cells is indicative of a cell proliferative disorder of pancreatic islet cells in the subject. As used herein, “predisposition” refers to an increased likely that an individual will have a disorder. Although a subject with a predisposition does not yet have the disorder, there exists an increased propensity to the disease.

The present invention also relates to a kit, which contains at least one oligonucleotide or polynucleotide that specifically binds to at least one gene as set forth in Tables 2 or 3 or a combination thereof. In one embodiment, a plurality of oligonucleotides of a kit of the invention includes at least one amplification primer pair (i.e., a forward primer and a reverse primer), and can include a plurality of amplification primer pairs, including. As such, a kit of the invention can contain, for example, one or a plurality of primer pairs specific for at least gene as set forth in Tables 2 and 3, such as IGFBP3 or fibronectin, for example.

The invention may also allow for primers to be generated which would include oligonucleotides of sufficient length and appropriate sequence so as to provide specific initiation of polymerization on a significant number of nucleic acids in the sample. Where the nucleic acid sequence of interest contains two strands, it is necessary to separate the strands of the nucleic acid before it can be used as a template for the amplification process. Strand separation can be effected either as a separate step or simultaneously with the synthesis of the primer extension products. This strand separation can be accomplished using various suitable denaturing conditions, including physical, chemical, or enzymatic means, the word “denaturing” includes all such means. One physical method of separating nucleic acid strands involves heating the nucleic acid until it is denatured. Typical heat denaturation may involve temperatures ranging from about 80 degrees to 105 degrees C. for times ranging from about 1 to 10 minutes. Strand separation may also be induced by an enzyme from the class of enzymes known as helicases or by the enzyme RecA, which has helicase activity, and in the presence of riboATP, is known to denature DNA. The reaction conditions suitable for strand separation of nucleic acids with helicases are described by Kuhn Hoffmann-Berling (CSH-Quantitative Biology, 43:63, 1978) and techniques for using RecA are reviewed in C. Radding (Ann. Rev. Genetics, 16:405-437, 1982).

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, generally at a pH of about 7-9. Preferably, a molar excess (for genomic nucleic acid, usually about 108:1 primer:template) of the two oligonucleotide primers is added to the buffer containing the separated template strands. It is understood, however, that the amount of complementary strand may not be known if the process of the invention is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. a large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90 degrees-100 degrees C. from about 1 to 10 minutes, preferably from 1 to 4 minutes. After this heating period, the solution is allowed to cool to approximately room temperature, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40 degrees C. Most conveniently the reaction occurs at room temperature.

The agent for polymerization may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, polymerase muteins, reverse transcriptase, and other enzymes, including heat-stable enzymes (i.e., those enzymes which perform primer extension after being subjected to temperatures sufficiently elevated to cause denaturation such as Taq DNA polymerase, and the like). Suitable enzymes will facilitate combination of the nucleotides in the proper manner to form the primer extension products which are complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

Preferably, the method of amplifying is by PCR, as described herein and as is commonly used by those of ordinary skill in the art. However, alternative methods of amplification have been described and can also be employed. PCR techniques and many variations of PCR are known. Basic PCR techniques are described by Saiki et al. (1988 Science 239:487-491) and by U.S. Pat. Nos. 4,683,195, 4,683,202 and 4,800,159, which are incorporated herein by reference.

The conditions generally required for PCR include temperature, salt, cation, pH and related conditions needed for efficient copying of the master-cut fragment. PCR conditions include repeated cycles of heat denaturation (i.e. heating to at least about 95 degrees C.) and incubation at a temperature permitting primer: adaptor hybridization and copying of the master-cut DNA fragment by the amplification enzyme. Heat stable amplification enzymes like the pwo, Thermus aquaticus or Thermococcus litoralis DNA polymerases which eliminate the need to add enzyme after each denaturation cycle, are commercially available. The salt, cation, pH and related factors needed for enzymatic amplification activity are available from commercial manufacturers of amplification enzymes.

As provided herein an amplification enzyme is any enzyme which can be used for in vitro nucleic acid amplification, e.g. by the above-described procedures. Such amplification enzymes include pwo, Escherichia coli DNA polymerase I, Klenow fragment of E. coli DNA polymerase I, T4 DNA polymerase, T7 DNA polymerase, Thermus aquaticus (Taq) DNA polymerase, Thermococcus litoralis DNA polymerase, SP6 RNA polymerase, T7 RNA polymerase, T3 RNA polymerase, T4 polynucleotide kinase, Avian Myeloblastosis Virus reverse transcriptase, Moloney Murine Leukemia Virus reverse transcriptase, T4 DNA ligase, E. coli DNA ligase or Q.beta. replicase. Preferred amplification enzymes are the pwo and Taq polymerases. The pwo enzyme is especially preferred because of its fidelity in replicating DNA.

Once amplified, the nucleic acid can be attached to a solid support, such as a membrane, and can be hybridized with any probe of interest, to detect any nucleic acid sequence. Several membranes are known to one of skill in the art for the adhesion of nucleic acid sequences. Specific non-limiting examples of these membranes include nitrocellulose (NITROPURE) or other membranes used in for detection of gene expression such as polyvinylchloride, diazotized paper and other commercially available membranes such as GENESCREEN, ZETAPROBE (Biorad), and NYTRAN Methods for attaching nucleic acids to these membranes are well known to one of skilled in the art. Alternatively, screening can be done in a liquid phase.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42 degrees C. (moderate stringency conditions); and 0.1×SSC at about 68 degrees C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically. In general, conditions of high stringency are used for the hybridization of the probe of interest.

The probe of interest can be detectably labeled, for example, with a radioisotope, a fluorescent compound, a bioluminescent compound, a chemiluminescent compound, a metal chelator, or an enzyme. Those of ordinary skill in the art will know of other suitable labels for binding to the probe, or will be able to ascertain such, using routine experimentation.

The following examples are intended to illustrate but not limit the invention.

### EXAMPLE 1

### Material and Methods

Selection of PEN samples

Permission for this study was obtained through The Johns Hopkins IRB (Joint Committee on Clinical Investigation). Snap-frozen tissue was obtained from eight pancreaticoduodenectomy resections at The Johns Hopkins Hospital performed for well-differentiated PENs. The clinicopathologic features of these eight cases are listed in Table 1. There were four males and four females in the cohort; the median age was 46 years (range 42-55 years). The median tumor size was 3.6 cm (range 2.0-6.0 cm). Two patients had concurrent lymphatic metastases; none had associated hepatic metastases. No patient had an associated endocrine hypersecretion syndrome, and all patients were alive with no evidence of disease at most recent follow up. Tumor samples were collected within 10 minutes of surgical resection, snap-frozen in liquid nitrogen, and stored at −80° C. Hematoxylin and eosin-stained sections from adjacent frozen tissue were prepared to confirm the diagnosis 2 and assess neoplastic cellularity. RNA was extracted from PENs containing>80% neoplastic cells and <10% necrosis on frozen section examination.

Human Islet Isolation

Since the normal bulk pancreas contains less than 2% islets of Langerhans, we used three enriched islet cell samples from cadaveric human pancreata as a normal control; islets were used for research purposes only when insufficient numbers were obtained for transplantation. Cadaveric human pancreata were obtained through the local organ procurement organization affiliated with the University of Pennsylvania School of Medicine (a JDRF islet cell distribution center); to the best of our knowledge, these individuals did not suffer from endocrine pancreatic pathology. Pancreatic islets were isolated using a modification of the automated Ricordi method ¹¹. In brief, collagenase (Liberase, Roche, Nutley, N. J.) at a concentration of 1.66 mg/ml in Hank's balanced salt solution (HBSS) was infused into the main pancreatic duct using a hand-held syringe and a Webster cannula. Organs were digested at 37 C for 15-25 minutes in Ricordi chamber, which was agitated with a mechanical shaker to facilitate the digestion process. After digestion, the dispersed pancreatic tissues were washed three times with RPMI culture medium and re-suspended in UW solution for one hour. Then, the liberated islets were separated from exocrine tissues using the COBE 2991 and a top-loaded continuous ficoll gradient (density range 1.055-1.120). The isolation process was performed without xenogeneic serum. Islet fractions with highest purity (>80-90%) were snap-frozen for RNA extraction and microarray analysis.

RNA Extraction and Hybridization

Sample preparation and processing procedure were performed at the Roswell Park Cancer Institute Microarray Core Facility, as described in the Affymetrix GeneChip® Expression Analysis Manual (Affymetrix Inc., Santa Clara, Calif.). Briefly, frozen PEN or islet samples were crushed in TRIzol (Invitr\dot{o}gen Inc., Carlsbad, Calif.) by using a Polytron homogenizer (Brinkman Instruments, Westbury, N.Y.). Total RNA was then extracted from the crushed tissue and cleaned using RNeasy™ columns according to manufacturer's protocol (Qiagen Inc., Valencia, Calif.). The integrity of total RNA was confirmed in each case using the Agilent 2100 Bioanalyzer (Agilent Technologies, Palo Alto, Calif.). Using 5-40 μg of total RNA, double-stranded cDNA was synthesized following SuperScript Choice system (Invitrogen Inc.). T7-(dT24) oligomer was used for priming the first-strand cDNA synthesis. The resultant cDNA was purified using Phase Lock Gel, phenol/chloroform extraction and precipitated with ethanol. The cDNA pellet was collected and dissolved in appropriate volume. Using cDNA as template, cRNA was synthesized using a T7 MegaScript In-Vitro Transcription (IVT) Kit (Ambion, Austin, Tex.). Biotinylated-11-CTP and 16-UTP ribonucleotides (Enzo Diagnostics Inc., Farmingdale, N.Y.) were added to the reaction as labeling reagents. IVT reactions were carried out at 37° C. for 6 hours and, the labeled cRNA obtained was purified using RNeasy columns (Qiagen Inc.). The cRNA was fragmented in fragmentation buffer (40 mM Tris-Acetate, pH 8.1, 100 mM KOAc, 30 mM MgOAc) for 35 minutes at 94° C. Fragmented cRNA (10-11 μg/probe array) was used to hybridize to human U133A GeneChip® array at 45° C. for 24 hours in a hybridization oven with constant rotation (60 rpm). The chips were washed and stained using Affymetrix fluidics stations. Staining was performed using streptavidin phycoerythrin conjugate (SAPE; Molecular Probes, Eugene, Oreg.), followed by the addition of biotinylated antibody to streptavidin (Vector Laboratories, Burlingame, Calif.), and finally with streptavidin phycoerythrin conjugate. Probe arrays were scanned using fluorometric scanners (Hewlett Packard Gene Array Scanner; Hewlett Packard Corporation, Palo Alto, Calif.). The scanned images were inspected and analyzed using established quality control measures.

Data Filtering and Analysis

The 11 .CEL files (3 normal islets, 8 PENs) generated by the Affymetrix Microarray Suite (MAS) version 5.0 were converted into .DCP files using dCHIP (www.dCHIP.org), as described previously by Li and Wong¹². The 11 .DCP files were normalized, and raw gene expression data generated using the dCHIP system of model-based analysis. For comparison of global gene expression profiles between normal islet cells and PENs, the three islets were designated as “baseline” (B), and the 8 PENs designated as “experiment” (E) in the dCHIP comparison software. Genes overexpressed 3-fold or higher in the PENs versus normal islets were then identified by defining the appropriate filtering criteria in the dCHIP software (mean E/mean B>3; mean E-mean B=100, P<0.05, t-test); similarly, genes underexpressed were identified by reversing the filtering criteria (mean B/mean E>3; mean B-mean E=100, P<0.05, t-test).

Immunohistochemistry

PEN tissue microarrays were generated from 53 archival paraffin-embedded well-differentiated PENs, as previously described¹³. Each cancer specimen was represented by one to five 1.4 mm cores on the tissue microarray, and when available, adjacent normal pancreas parenchyma containing normal islets of Langerhans were also arrayed for comparison. In summary, the 53 arrayed PENs consisted of 44 primary tumors (9 with paired lymph node metastases), and 9 isolated hepatic metastases without corresponding primary tumor; adjacent normal pancreatic parenchyma was arrayed in 43/44 primary PENs.

Slides were deparaffinized in fresh xylene and rehydrated through sequential graded ethanol steps. Antigen retrieval was performed by citrate buffer incubation (18 mM citric acid, 8.2 mM sodium citrate, pH 6.0) using a household vegetable steamer (Black and Decker) for 60 minutes. Slides were incubated for 5 minutes with 3% hydrogen peroxide, washed in TBS/T (20 mM Tris, 140 mM NaCl, 0.1% Tween-20, pH 7.6), and incubated in appropriate antibody dilutions for p21(CDKN1A) (Dako, Carpinteria, Calif.; 1:75), CD99 (013) (Signet, Dedham, Mass.; 1:400), insulin-like growth factor binding protein 3 (IGFBP3) (Santa Cruz Biotechnology, Santa Cruz, Calif.; 1:40), and fibronectin (Dako; 1:1200) for 60 minutes at room temperature. Normal saline was substituted for the primary antibody in control sections. The avidin-biotin-peroxidase complex method from DAKO (Glostrup, Denmark) was used and slides were subsequently counterstained with hematoxylin. Assessment of immunohistochemical labeling in the tissue microarrays was performed by two of the authors (D.E.H. and A. M.). Loss of nuclear p21 or membranous CD99 in>95% of neoplastic endocrine cells was considered as “negative”; similarly, labeling of cytoplasmic IGFBP3 or membranous/cytoplasmic fibronectin in>25% of neoplastic cells was considered “positive”. Since normal islets also express IGFBP3, cases were considered “positive” when the staining intensity was greater than in normal islets.

Statistical analysis was performed using the Analyze-it™ software package for Microsoft. Two-tailed Fisher exact tests were performed for determining differences in expression of candidate genes by immunohistochemistry, and a P value of <0.05 was considered significant.

### EXAMPLE 2

### Differentially Expressed Transcripts in PENs

Normalization and comparison of Affymetrix microarray hybridization data was performed using dCHIP¹² Sixty-six transcripts were significantly overexpressed 3-fold or higher in PENs compared to enriched islet cells (P<0.05, t-test), and a partial list of transcripts with known functions is illustrated in Table 2. The list of upregulated transcripts included putative oncogenes (MLLT10/AF10)^{14,15}, growth factors (insulin like-growth factor binding protein 3 [IGFBP3])^{16,17}, cell adhesion and migration molecules (fibronectin)¹⁸⁻²⁰, and endothelial elements (MUC18/MelCAM, CD31)²¹. Similarly, 119 transcripts were significantly underexpressed 3-fold or less in PENs compared to enriched islet cells (P<0.05, t-test), and a partial list of transcripts with known functions is illustrated in Table 3. The list of downregulated transcripts included cell cycle checkpoint proteins (p21/Cip1)^{22,23}, the MIC2 (CD99) cell surface glycoprotein²⁴, genes involved in DNA damage repair and genomic stability (O-6-methylguanine-DNA methyltransferase, GADD45)^{25,26}, putative metastasis suppressor genes (NME3/DR-nme23) 27, andjunD, a MEN1-regulated transcription factor 28. The complete list of over- and underexpressed transcripts is available from the authors on request. A literature search of PubMed (www.ncbi.nlm.nih.gov/PubMed) revealed that the differential expression of several genes in this study has previously been reported—either singly or through global microarray expression analyses—in other cancer types, in principle validating our approach. A second PubMed search using the gene name and either “islet cell” or “pancreatic endocrine” only yielded two genes (p21/CDKN1A and CD99) that have been previously reported as underexpressed in PENs, by immunohistochemistry^{29,30}. Thus, the overwhelming majority of the 185 differentially expressed genes reported in this study represent newly described tumor markers for PENs.

### EXAMPLE 3

### Validation of Selected Differentially Expressed Genes in Tissue Microarrays

Four differentially expressed genes, two downregulated (p21 and CD99) and two upregulated (IGFBP3 and fibronectin), were validated by immunohistochemistry using PEN tissue microarrays (Table 4). Table 4 also lists the labeling pattern seen in normal islets as a comparison. The denominator (i.e., total numbers of cases evaluated) is variable between the antibodies because of “dropout” of tissue cores during the TMA staining process.

Normal islet cells demonstrated robust nuclear p21 expression, with labeling seen in 10-25% of nuclei within a given islet. Fifty-two cases could be evaluated for p21 labeling on the PEN TMA. Overall, loss of p21 expression (i.e., <5% nuclear p21 labeling) was seen 24/52 (46%) PENs, including 21/44 (48%) primary tumors and 3/8 (38%) isolated hepatic metastases. In 7/8 (88%) lymph node metastases, the pattern of p21 expression was concordant with the corresponding primary tumor, i.e., 3/7 matched lesions retained p21, while labeling was absent in the remaining four. In only one case, the primary tumor labeled with p21, while the matched nodal metastasis lacked p21 expression. There were no statistically significant differences in loss of p21 expression between primary (48%) and all metastatic PEN lesions (7/15 or 47%) (P=1.0, Fisher exact test).

Fifty-three cases could be evaluated for CD99 labeling. Intense, diffuse membranous CD99 expression was seen in normal islet cells, while as many as 48/53 (91%) PENs, including 39/44 (89%) primary tumors and 9/9 (100%) isolated hepatic metastases, demonstrated loss of CD99 expression. CD99 expression was concordant in 9/9 (100%) paired primary and lymph node metastasis on the PEN tissue array, with loss of CD99 expression demonstrable in 8/9 matched lesions, and retention of labeling in 1 case. There were no statistically significant differences in loss of CD99 expression between primary (91%) and all metastatic PEN lesions (17/18 or 94%) (P=0.86, Fisher exact test).

IGFBP3 and fibronectin were upregulated in PENs compared to normal islets on microarray analysis (4.1 and 3.9 fold, respectively). Forty-seven cases could be evaluated for IGFBP3 labeling on the PEN TMA. Immunohistochemical analysis demonstrated weak to moderate granular IGFBP3 labeling in the cytoplasm of almost all normal islets. Using the staining intensity of normal islets as a baseline, 14/47 (30%) cases expressed IGFBP3 equal to or less than the normal islet cells. In contrast, intense, diffuse overexpression of IGFBP3 was seen in 33/47 (70%) of PENs, including 24/40 (60%) primary PENs, and 7/7 (100%) isolated hepatic metastases. IGFBP3 expression was concordant in 9/9 (100%) paired primary and lymph node metastasis on the PEN TMA with 8/9 (89%) cases co-expressing IGFBP3, and one matched primary and nodal metastasis demonstrating no labeling. There was a statistically significant difference in IGFBP3 overexpression in all metastatic lesions (15/16 or 93%) versus primary PENs (24/40 or 60%) (P=0.022, Fisher exact test). In addition, IGFBP3 overexpression in a primary PEN was more common in the presence of synchronous metastases; 8/10 or 80% of primary PENs with synchronous metastases overexpressed IGFBP3 versus 16/30 or 53% of PENs that were localized to the pancreas; however, the difference was not statistically significant (P=0.26, Fisher exact test).

Fibronectin is traditionally considered a stromal marker, and expectedly, intense fibronectin expression was present in regions of peritumoral desmoplasia (not illustrated), while normal islet cells were negative. Of note however, diffuse, membranous labeling of the neoplastic cells themselves was present in a significant minority, 11/50 (22%) cases; the latter included 10/42 (24%) primary tumors and 1/8 (13%) isolated hepatic metastasis. Another notable feature of fibronectin labeling was its propensity to be upregulated in lymphatic metastases compared to the paired primary tumor; thus, of 9 paired primary tumor and lymph node metastases examined, 5/9 (55%) demonstrated labeling that was restricted to the metastasis, while the primary lesion was negative. There was no significant difference in fibronectin overexpression between primary (24%) and all metastatic PEN lesions (6/17 or 35%) (P=0.55, Fisher exact test); in contrast, the difference in fibronectin expression between nodal metastases alone (55%) and primary PENs demonstrated a trend, but did not reach statistical significance (P=0.14, Fisher exact test).

Well-differentiated pancreatic endocrine neoplasms (PENs), unlike their universally aggressive exocrine counterpart, are comprised of distinct malignant and indolent subsets of tumors. As a consequence, it is imperative to identify, and aggressively treat, PENs likely to result in adverse clinical outcomes. Characterization of the changes in gene expression and cellular pathway activation that contribute to carcinogenesis of the endocrine pancreas may identify markers for early detection, facilitate accurate prognostic classification of PENs, and lead to new molecular targets for therapy.

The present invention shows the first global gene expression analysis of PENs and compared this to the gene expression in highly enriched isolated human islets, using the second generation Affymetrix U133 platform, containing ˜22,000 human transcripts. We have identified 66 transcripts overexpressed 3-fold or higher and 119 transcripts expressed 3-fold or lower in PENs compared to normal islet cells. In addition, we have immunohistochemically validated the differential expression of four genes in TMAs comprised of archival primary and metastatic PEN lesions. A search on the National Library of Medicine's PubMed (www.ncbi.nlm.nih.gov/pubmed) using the gene name and either “pancreatic endocrine” or “islet cell” revealed that only two transcripts in our combined list of 185 genes—p21 and CD99—has been previously described in the context of PENs^{29,30}. Thus, the overwhelming majority of genes we have identified represent novel differentially expressed genes in PENs.

A particular strength of this study is the use of enriched human islet cells as a normal control, as opposed to bulk pancreas, which contains <2% islets of Langerhans. This approach has permitted an accurate comparison of neoplastic endocrine cells with their normal counterpart, instead of a plethora of non-endocrine tissues. We are aware that this analysis may have generated a minority of “false positive” differentially expressed genes (for example, genes present in tumor-associated endothelium or stromal elements), which would not be expressed by enriched islet cell extracts. Nevertheless, by judiciously parsing the gene lists using known gene function and cellular localization, and more importantly, by validation in tissue sections, we have generated a rational expression profile of neoplastic endocrine cells.

The paramount importance of tissue validation of microarray expression data is best exemplified by two transcripts in the overexpressed gene list: fibronectin and melanoma cell adhesion molecule (MelCAM or Muc18). Fibronectin is classically considered a stromal marker⁹. Yet, on immunohistochemical analysis, a significant minority (22%) of PENs expressed fibronectin on the neoplastic cell surface, a proportion that was even higher in nodal metastasis (see below, page 16). On the contrary, MelCAM/Muc18, a cell adhesion molecule otherwise present in several cancer types 31-33, was not expressed in any of the PEN cases on our TMA. MelCAM/Muc18 labeling was restricted to the endothelium only^{33,34}, and this transcript was presumably “overexpressed” in PENs due to absence of vascular structures in enriched islet cell extracts. The ensuing discussion will address some of the genes whose differential expression we have validated in tissue sections of PENs.

As previously stated, we confirmed the differential expression of four genes using PEN TMA. This includes two downregulated (p21 and CD99) and two upregulated genes (IGFBP3 and fibronectin), respectively. The loss of p21, a cyclin dependent kinase inhibitor, has been reported in a large number of human neoplasms, including PENs^{22,23}. In a study of 109 gastrointestinal carcinoid tumors, including 42 PENs, Canavese et al found low level of p21 expression in most well differentiated PENs³⁰. The authors used a “p21 labeling index”, and reported a median labeling index of 1.29% for “benign tumors” (range 0-20.48%). Since the scoring criterion was different from the present TMA-based study, we cannot make a direct comparison. In contrast, Choi et al reported loss ofp21 in 40% of non-functional PENs in their series 29, which is in excellent agreement with our own findings (46% loss of expression). In our series of PENs, we failed to find a statistically significance difference in p21 expression between primary and metastatic PEN lesions, and p21 expression was concordant in ˜90% of matched primary pancreatic and nodal metastatic lesions.

In summary, we have performed the first global expression profiling of PENs, and have identified 119 significantly downregulated and 66 significantly upregulated genes compared to enriched human pancreatic islet cells, using the Affymetrix U133 platform. We have validated the differential expression for a subset of these genes using TMAs, and have identified overexpression of IGFBP3 as a potential predictor of metastases, and fibronectin as a potential target for therapy in a subset of PEN lesions, particularly those with nodal metastases.

TABLE 1
Clinico-pathologic characteristics of eight patients with PEN

| Tumor | Age | Sex | Size (cm) | IHC | LN status | Outcome |
|---|---|---|---|---|---|---|
| PEN 1 | 54 | F | 6.5 | I, Gl, Ga, | 0/12 | NED |
| | | | | Soma− | | |
| PEN 2 | 47 | F | 2.0 | I, Gl, Ga, | 1/16 | NED |
| | | | | Soma− | | |
| PEN 3 | 55 | F | 3.7 | Soma+; I, | 0/12 | NED |
| | | | | Gl, Ga− | | |
| PEN 4 | 52 | M | 4.6 | Not done | 0/12 | NED |
| PEN 5 | 45 | M | 6.0 | Not done | 0/8 | NED |
| PEN 6 | 42 | F | 2.0 | I, Gl, Ga, | 0/6 | NED |
| | | | | Soma− | | |
| PEN7 | 45 | M | 3.5 | Not done | 0/9 | NED |
| PEN8 | 45 | M | 3.0 | I, Gi, Ga, | 1/10 | NED |
| | | | | Soma− | | |

IHC = Immunohistochemistry, LN = lymph node, NED = no evidence of disease, I—Insulin, Gl = Glucagon, Ga = Gastrin, Soma = Somatostatin, + = positive labeling, − = negative labeling.
Note:
None of the patients had a known endocrine hypersecretion syndrome.

TABLE 2
Selected differentially overexpressed transcripts in PENs

| | Fold | | |
|---|---|---|---|
| Gene | change | P value | Function |
| Adrenergic, beta-3, receptor | 3.80 | 0.004 | Adrenergic receptor linked |
| | | | to diabetes and obesity. |
| CDC5 cell division cycle 5-like (S. pombe) | 5.30 | 0.008 | Cell cycle molecule; |
| | | | promotes G2-M progression |
| ERGL protein (ERGIC-53-like | 3.99 | 0.005 | Transmembrane protein |
| protein) | | | expressed in normal and |
| | | | neoplastic prostate |
| Fibronectin 1 | 3.99 | 0.005 | Cell surface adhesion and |
| | | | migration molecule; stromal |
| | | | desmoplasia |
| Insulin-like growth factor binding | 4.11 | 0.005 | IGF binding protein; has |
| protein 3 (IGFBP3) | | | independent growth- |
| | | | potentiating effects |
| Melanoma cell adhesion molecule | 3.00 | 0.019 | Cell adhesion molecule |
| (MelCAM/MUC18) | | | found in cancer cells of |
| | | | neuroectodermal lineage; |
| | | | endothelial molecule |
| Meprin A, beta | 4.91 | 0.006 | Metalloprotease; associated |
| | | | with cancer invasion |
| Trithorax homolog, Drosophila; | 3.79 | 0.023 | Fusion partner of MLL gene |
| translocated to, 10 | | | on chromosome 10 |
| (MLLT10/AF10) | | | |
| Secreted protein, acidic, cysteine- | 3.211 | 0.019 | Tumor-associated |
| rich (osteonectin) | | | glycoprotein; may |
| | | | contribute to invasive |
| | | | phenotype |
| Zinc finger protein 43 (HTF6) | 3.31 | 0.007 | Transcription factor |

TABLE 3
Selected differentially underexpressed transcripts in PENs

| | Fold | | |
|---|---|---|---|
| Gene | change | P value | Function |
| Anti-oxidant protein 2 (AOP2) | −4.18 | 0.0001 | Thiol-specific anti-oxidant |
| B-cell CLL/lymphoma 7C | −3.31 | 0.009 | Recurrent breakpoint in high- |
| | | | grade non-Hodgkin lymphoma |
| Cyclin-dependent kinase inhibitor | −5.18 | 0.039 | Negative regulator of cell cycle |
| 1A (p21, Cip1) | | | |
| Growth arrest and DNA-damage | −3.30 | 0.011 | Checkpoint protein involved in |
| inducible, alpha | | | DNA damage repair |
| Heat shock protein, 90kD, alpha | −3.01 | 0.0008 | Stabilizes variety of cellular |
| | | | proteins, including CDK4 |
| JunD proto-oncogene | −7.56 | 0.010 | Target of menin; immortalized |
| | | | cells lacking JunD demonstrate |
| | | | increased proliferation |
| MIC2 (CD99) | −3.34 | 0.022 | Cell surface glycoprotein |
| Non-metastatic cells 3, protein | −4.75 | 0.021 | Metastasis suppressor and |
| expressed in | | | promoter of differentiation |
| SCAN domain-containing 1 | −3.44 | 0.0001 | Transcriptional regulator |

TABLE 4
Validation of selected differentially expressed genes
in tissue microarrays

| | | Abnormal | Abnormal |
|---|---|---|---|
| | | Expression | Expression |
| | | Pattern in | Pattern in |
| | Abnormal Expression | Primary | Metastatic |
| | Pattern in PENs* | PENs* | PENs* |
| Antigen | (N = 53)Ψ | (N = 44)Ψ | (N = 18)Ψ |
| p21 | 24/52 (46%) | 21/44 (48%) | 7/15 (47%) |
| CD99 | 48/53 (91%) | 39/44 (89%) | 17/18 (94%) |
| IGFBP3 | 33/47 (70%) | 24/40 (60%)# | 15/16 (93%)# |
| Fibronectin | 11/50 (22%) | 10/42 (24%) | 6/17 (35%) |
| *Abnormal expression pattern denotes loss of labeling for p21 and CD99, and overexpression for IGFBP3 and fibronectin. The criteria for immunohistochemical scoring are detailed in the text. | | | |
| ΨTotal number of cases evaluated (i.e., possible maximum denominator) is 53; this includes 44 primary PEN lesions and 9 isolated hepatic metastases. In addition, 9 matched nodal metastases were also evaluated, such that the total number of metastases (i.e., | | | |
| # possible maximum denominator) in this series is 18. As explained in the text, the denominators are variable because of “dropout” during immunohistochemical staining of the tissue microarrays. | | | |

#Difference is statistically significant (P = 0.022, Fisher exact test)

### REFERENCES

1. Solcia E, Rindi G, Paolotti D, La Rosa S, Capella C, Fiocca R: Clinicopathological profile as a basis for classification of the endocrine tumours of the gastroenteropancreatic tract. Ann Oncol 1999, 10 Suppl 2:S9-15
2. Solcia E, Kloppel G, Sobin L H: Histological Typing of Endocrine Tumors. New York, Springer-Verlag, 2001
3. Phan G Q, Yeo C J, Hruban R H, Lillemoe K D, Pitt H A, Cameron J L: Surgical experience with pancreatic and peripancreatic neuroendocrine tumors: review of 125 patients. J Gastrointest Surg 1998, 2:472-482
4. Kloppel G, Solcia E, Capella C, Heitz P U: Classification of neuroendocrine tumours. Ital J Gastroenterol Hepatol 1999, 31 Suppl 2:S111-116
5. La Rosa S, Sessa F, Capella C, Riva C, Leone B E, Klersy C, Rindi G, Solcia E: Prognostic criteria in nonfunctioning pancreatic endocrine tumours. Virchows Arch 1996, 429:323-333
6. Lee C S, Charlton I G, Williams R A, Dhillon A P, Rode J: Malignant potential of aneuploid pancreatic endocrine tumours. J Pathol 1993, 169:451-456
7. Rigaud G, Missiaglia E, Moore P S, Zamboni G, Falconi M, Talamini G, Pesci A, Baron A, Lissandrini D, Rindi G, Grigolato P, Pederzoli P, Scarpa A: High resolution allelotype of nonfunctional pancreatic endocrine tumors: identification of two molecular subgroups with clinical implications. Cancer Res 2001, 61:285-292
8. Gumbs A A, Moore P S, Falconi M, Bassi C, Beghelli S, Modlin I, Scarpa A: Review of the clinical, histological, and molecular aspects of pancreatic endocrine neoplasms. J Surg Oncol 2002, 81:45-53; discussion 54
9. Iacobuzio-Donahue C, Maitra A, Olsen M, Lowe A, van Heek NT, Rosty C, Walter K, Sato N, Parker A, Ashfaq R, Jaffee E, Ryu B, Jones J, Eshelman J, Yeo C J, Cameron J L, Kern S E, Hruban R H, Brown P O, Goggins M: Exploration of global expression patterns in pancreatic adenocarcinomas using cDNA microarrays. Am J Pathol 2003, 162:1151-1162
10. Iacobuzio-Donahue C A, Maitra A, Shen-Ong G L, van Heek T, Ashfaq R, Meyer R, Walter K, Berg K, Hollingsworth M A, Cameron J L, Yeo C J, Kern S E, Goggins M, Hruban R H: Discovery of novel tumor markers of pancreatic cancer using global gene expression technology. Am J Pathol 2002, 160:1239-1249
11. Markmann J F, Deng S, Huang X, Velidedeoglu E, Desai N M, Wolf B, Matschinsky F, Barker F, Naji A: Successful human islet transplantation from single donors. Ann Surg 2003, In Press
12. Li C, Wong W H: Model-based analysis of oligonucleotide arrays: expression index computation and outlier detection. Proc Natl Acad Sci USA 2001, 98:31-36
13. Luo J, Zha S, Gage W R, Dunn T A, Hicks J L, Bennett C J, Ewing C M, Platz E A, Ferdinandusse S, Wanders R J, Trent J M, Isaacs W B, De Marzo A M: Alpha-methylacyl-CoA racemase: a new molecular marker for prostate cancer. Cancer Res 2002, 62:2220-2226
14. Debernardi S, Bassini A, Jones L K, Chaplin T, Linder B, de Bruijn D R, Meese E, Young B D: The MLL fusion partner AF10 binds GAS41, a protein that interacts with the human SWI/SNF complex. Blood 2002, 99:275-281
15. Chaplin T, Ayton P, Bernard O A, Saha V, Della Valle V, Hillion J, Gregorini A, Lillington D, Berger R, Young B D: A novel class of zinc finger/leucine zipper genes identified from the molecular cloning of the t(10;11) translocation in acute leukemia. Blood 1995, 85:1435-1441
16. Furstenberger G, Senn H J: Insulin-like growth factors and cancer. Lancet Oncol 2002, 3:298-302
17. Vadgama JV, Wu Y, Datta G, Khan H, Chillar R: Plasma insulin-like growth factor-I and serum IGF-binding protein 3 can be associated with the progression of breast cancer, and predict the risk of recurrence and the probability of survival in African-American and Hispanic women. Oncology 1999, 57:330-340
18. Danen E H, Yamada K M: Fibronectin, integrins, and growth control. J Cell Physiol 2001, 189:1-13
19. Ruoslahti E: Fibronectin and its integrin receptors in cancer. Adv Cancer Res 1999, 76:1-20
20. Labat-Robert J: Fibronectin in malignancy. Semin Cancer Biol 2002, 12:187-195
21. Shih IM: The role of CD146 (Mel-CAM) in biology and pathology. J Pathol 1999, 189:4-11
22. Waldman T, Kinzler K W, Vogelstein B: p21 is necessary for the p53-mediated G1 arrest in human cancer cells. Cancer Res 1995, 55:5187-5190
23. Bunz F, Dutriaux A, Lengauer C, Waldman T, Zhou S, Brown J P, Sedivy J M, Kinzler K W, Vogelstein B: Requirement for p53 and p21 to sustain G2 arrest after DNA damage. Science 1998, 282:1497-1501
24. Gelin C, Aubrit F, Phalipon A, Raynal B, Cole S, Kaczorek M, Bernard A: The E2 antigen, a 32 kd glycoprotein involved in T-cell adhesion processes, is the MIC2 gene product. Embo J 1989, 8:3253-3259
25. Esteller M, Garcia-Foncillas J, Andion E, Goodman S N, Hidalgo O F, Vanaclocha V, Baylin S B, Herman J G: Inactivation of the DNA-repair gene MGMT and the clinical response of gliomas to alkylating agents. N Engl J Med 2000, 343:1350-
26. Tran H, Brunet A, Grenier J M, Datta S R, Formace A J, Jr., DiStefano P S, Chiang L W, Greenberg M E: DNA repair pathway stimulated by the forkhead transcription factor FOXO3a through the Gadd45 protein. Science 2002, 296:530-534
27. Venturelli D, Martinez R, Melotti P, Casella I, Peschle C, Cucco C, Spampinato G, Darzynkiewicz Z, Calabretta B: Overexpression of DR-nm23, a protein encoded by a member of the nm23 gene family, inhibits granulocyte differentiation and induces apoptosis in 32Dc13 myeloid cells. Proc Natl Acad Sci USA 1995, 92:7435-7439
28. Weitzman J B, Fiette L, Matsuo K, Yaniv M: JunD protects cells from p53-dependent senescence and apoptosis. Mol Cell 2000, 6:1109-1119
29. Choi Y L, Oh Y L, Kim S H, Park C K, Ahn G: Comparative study of non-functional islet cell tumors and pancreatic solid and papillary neoplasms: biological behavior and immunohistochemistry. Pathol Int 2002, 52:358-366
30. Canavese G, Azzoni C, Pizzi S, Corleto VD, Pasquali C, Davoli C, Crafa P, Delle Fave G, Bordi C: p27: a potential main inhibitor of cell proliferation in digestive endocrine tumors but not a marker of benign behavior. Hum Pathol 2001, 32:1094-1101
31. Wu G J, Varma V A, Wu M W, Wang S W, Qu P, Yang H, Petros J A, Lim S D, Amin M B: Expression of a human cell adhesion molecule, MUC18, in prostate cancer cell lines and tissues. Prostate 2001, 48:305-315
32. Johnson JP: Cell adhesion molecules in the development and progression of malignant melanoma. Cancer Metastasis Rev 1999, 18:345-357
33. Shih IM, Wang TL, Westra WH: Diagnostic and biological implications of mel-CAM expression in mesenchymal neoplasms. Clin Cancer Res 1996, 2:569-575
34. Sers C, Riethmuller G, Johnson J P: MUC18, a melanoma-progression associated molecule, and its potential role in tumor vascularization and hematogenous spread. Cancer Res 1994, 54:5689-5694
35. Goodfellow P J, Darling S M, Thomas N S, Goodfellow P N: A pseudoautosomal gene in man. Science 1986, 234:740-743
36. Ambros I M, Ambros P F, Strehl S, Kovar H, Gadner H, Salzer-Kuntschik M: MIC2 is a specific marker for Ewing's sarcoma and peripheral primitive neuroectodermal tumors. Evidence for a common histogenesis of Ewing's sarcoma and peripheral primitive neuroectodermal tumors from MIC2 expression and specific chromosome aberration. Cancer 1991, 67:1886-1893
37. Movahedi-Lankarani S, Hruban R H, Westra W H, Klimstra D S: Primitive neuroectodermal tumors of the pancreas: a report of seven cases of a rare neoplasm. Am J Surg Pathol 2002, 26:1040-1047
38. Maitra A, Weinberg A G, Schneider N, Patterson K: Detection of t(11;22)(q24;q 12) translocation and EWS-FLI-1 fusion transcript in a case of solid pseudopapillary tumor of the pancreas. Pediatr Dev Pathol 2000, 3:603-605
39. Pelosi G, Fraggetta F, Sonzogni A, Fazio N, Cavallon A, Viale G: CD99 immunoreactivity in gastrointestinal and pulmonary neuroendocrine tumours. Virchows Arch 2000, 437:270-274
40. Kim S H, Shin Y K, Lee I S, Bae Y M, Sohn H W, Suh Y H, Ree H J, Rowe M, Park SH: Viral latent membrane protein 1 (LMP-1)-induced CD99 down-regulation in B cells leads to the generation of cells with Hodgkin's and Reed-Sternberg phenotype. Blood 2000, 95:294-300
41. Lee I, Kim M K, Choi E Y, Mehl A, Jung K C, Gil M C, Rowe M, Park S H: CD99 expression is positively regulated by Sp1 and is negatively regulated by Epstein-Barr virus latent membrane protein 1 through nuclear factor-kappaB. Blood 2001, 97:3596-3604
42. Shen L, Dean N M, Glazer R I: Induction of p53-dependent, insulin-like growth factor-binding protein-3-mediated apoptosis in glioblastoma multiforme cells by a protein kinase Calpha antisense oligonucleotide. Mol Pharmacol 1999, 55:396-402
43. Collard T J, Guy M, Butt A J, Perks C M, Holly J M, Paraskeva C, Williams A C: Transcriptional upregulation of the insulin-like growth factor binding protein IGFBP-3 by sodium butyrate increases IGF-independent apoptosis in human colonic adenoma-derived epithelial cells. Carcinogenesis 2003, 24:393-401
44. Goodwin P J, Ennis M, Pritchard K I, Trudeau M E, Koo J, Hartwick W, Hoffma B, Hood N: Insulin-like growth factor binding proteins 1 and 3 and breast cancer outcomes. Breast Cancer Res Treat 2002, 74:65-76
45. Bittner M, Meltzer P, Chen Y, Jiang Y, Seftor E, Hendrix M, Radmacher M, Simon R, Yakhini Z, Ben-Dor A, Sampas N, Dougherty E, Wang E, Marincola F, Gooden C, Lueders J, Glatfelter A, Pollock P, Carpten J, Gillanders E, Leja D, Dietrich K, Beaudry C, Berens M, Alberts D, Sondak V: Molecular classification of cutaneous malignant melanoma by gene expression profiling. Nature 2000, 406:536-540
46. Clark E A, Golub T R, Lander E S, Hynes R O: Genomic analysis of metastasis reveals an essential role for RhoC. Nature 2000, 406:532-535
47. Humphries M J, Olden K, Yamada K M: A synthetic peptide from fibronectin inhibits experimental metastasis of murine melanoma cells. Science 1986, 233:467-470
48. Marty C, Odermatt B, Schott H, Neri D, Ballmer-Hofer K, Klemenz R, Schwendener R A: Cytotoxic targeting of F9 teratocarcinoma tumours with anti-ED-B fibronectin scFv antibody modified liposomes. Br J Cancer 2002, 87:106-112.

Although the invention has been described with reference to the above example, it will be understood that modifications and variations are encompassed within the spirit and scope of the invention. Accordingly, the invention is limited only by the following claims.

1. A method for diagnosing pancreatic endocrine neoplasm (PEN) in a subject comprising:
a) obtaining a nucleic acid sample from the subject;
b) contacting nucleic acids of the sample with an array comprising a plurality of DNAs under conditions to form one or more hybridization complexes;
c) detecting the hybridization complexes; and
d) comparing the levels of the hybridization complexes detected in step (c) with the level of hybridization complexes detected in a non-diseased sample, wherein an altered level of hybridization complexes detected in step (c) compared with the level of hybridization complexes of a non-diseased sample correlates with the presence of PEN in the subject.

2. The method of claim 1, wherein the DNAs are immobilized on a substrate.

3. The method of claim 1, wherein the DNAs are hybridizable elements in a micro array.

4. The method of claim 1, wherein the DNA is cDNA.

5. The method of claim 1, wherein the nucleic acid from the subject comprises at least one nucleic acid sequence selected from Table 2 or Table 3 and combinations thereof.

6. The method of claim 5, wherein the nucleic acid comprises a nucleic acid sequence encoding a putative oncogene, growth factor, cell adhesion and migration molecule, endothelial element, cell cycle checkpoint protein, metastasis suppressor gene, transcription factor, or a combination thereof.

7. The method of claim 1, wherein the at least one gene comprises MLLT10/AF10, IGFBP3, fibronectin, MUC18, p21/Cip1, GADD45, NME3, junD, or a combination thereof.

8. The method of claim 5, wherein the nucleic acid encodes fibronectin.

9. The method of claim 5, wherein the nucleic acid encodes IGFBP3.

10. A method for predicting the likelihood of metastases from a primary pancreatic endocrine neoplasm (PEN) in a subject comprising:
a) obtaining a nucleic acid sample from the subject;
b) contacting nucleic acids of the sample with a nucleic acid sequence that is complementary to IGFBP3 under conditions that allow formation of hybridization complexes;
c) detecting the hybridization complexes; and
d) comparing the levels of the hybridization complexes detected in step (c) with the level of hybridization complexes detected in a non-diseased sample, wherein an altered level of hybridization complexes detected in step (c) compared with the level of hybridization complexes of a non-diseased sample correlates with the likelihood of developing metastases in the subject.

11. A method for predicting the likelihood of metastases from a primary pancreatic endocrine neoplasm (PEN) in a subject comprising:
a) obtaining a nucleic acid sample from the subject;
b) contacting nucleic acids of the sample with a nucleic acid sequence that is complementary to fibronectin under conditions that allow formation of hybridization complexes;
c) detecting the hybridization complexes; and
d) comparing the levels of the hybridization complexes detected in step (c) with the level of hybridization complexes detected in a non-diseased sample, wherein an altered level of hybridization complexes detected in step (c) compared with the level of hybridization complexes of a non-diseased sample correlates with the likelihood of developing metastases in the subject.

12. A method for diagnosing pancreatic endocrine neoplasm (PEN) in a subject comprising:
a) obtaining a nucleic acid sample from the subject;
b) contacting nucleic acids of the sample with a nucleic acid sequence that is complementary to CD99 under conditions that allow formation of hybridization complexes;
c) detecting the hybridization complexes; and
d) comparing the levels of the hybridization complexes detected in step (c) with the level of hybridization complexes detected in a non-diseased sample, wherein an altered level of hybridization complexes detected in step (c) compared with the level of hybridization complexes of a non-diseased sample correlates with the presence of PEN in the subject.

13. A method for diagnosing pancreatic endocrine neoplasm (PEN) in a subject comprising:
a) obtaining a nucleic acid sample from the subject;
b) contacting nucleic acids of the sample with a nucleic acid sequence that is complementary to p21 under conditions that allow formation of hybridization complexes;
c) detecting the hybridization complexes; and
d) comparing the levels of the hybridization complexes detected in step (c) with the level of hybridization complexes detected in a non-diseased sample, wherein an altered level of hybridization complexes detected in step (c) compared with the level of hybridization complexes of a non-diseased sample correlates with the presence of PEN in the subject.

14. A method for monitoring the course of treatment in a subject having a pancreatic endocrine neoplasm (PEN) comprising:
a) obtaining a nucleic acid sample from the subject;
b) contacting nucleic acid of the sample with an array comprising a plurality of DNAs under conditions to form one or more hybridization complexes;
c) detecting said hybridization complexes; and
d) comparing the levels of the hybridization complexes detected in step (c) with the level of hybridization complexes detected in the subject prior to and during the course of treatment, wherein an altered level of hybridization complexes detected prior to and during treatment correlates with the effectiveness of treatment in the subject.

15. The method of claim 14, wherein the DNAs are immobilized on a substrate.

16. The method of claim 14, wherein the DNAs are hybridizable elements in a microarray.

17. The method of claim 14, wherein the DNA is cDNA.

18. The method of claim 14, wherein when fibronectin or IGFBP3 are detected, expression during the course of treatment is decreased as compared to prior to treatment.

19. The method of claim 14, wherein when CD99 or p21 are detected, expression during the course of treatment is increased as compared to prior to treatment.

20. A method for identifying a cell that exhibits or is predisposed to exhibiting unregulated growth, comprising detecting, in a test cell, at least one gene expressed differentially as compared to a normal islet cell, wherein the at least one gene is set forth in Table 2 or Table 3, or a combination thereof, thereby identifying the test cell as a cell that exhibits or is predisposed to exhibiting unregulated growth.

21. A method of identification of a compound that affects tumorigenesis or disease progression of a pancreatic endocrine neoplasm comprising hybridizing nucleic acid obtained from cells of PENs or cells suspected of being PENs or being at risk for developing into PENs with at least one gene to form a pattern of expression and comparing the pattern with a pattern obtained from hybridizing nucleic acid obtained from cells of PENs or cells suspected of being PENs or being at risk for developing into PENs following treatment with an agent or compound for treating PEN cells, wherein a difference in the patterns is indicative of an effect of the agent or compound on expression of genes, and thereby an affect on tumorigenesis or disease progression.

22. The method of claim 21, wherein the pattern of expression includes genes selected from Tables 2 or 3 or a combination thereof.

23. The method of claim 21, wherein the pattern of expression includes at least IGFBP3.

24. The method of claim 21, wherein the pattern of expression includes at least fibronectin.

25. A method of treating a pancreatic endocrine neoplasm in a subject comprising contacting islet cells of the subject with a compound or agent that affects expression of one or more genes associated with the neoplasm, wherein the one or more gene is selected from a gene set forth in Table 2 or 3 or a combination thereof.

26. The method of claim 25, wherein the affect is upregulation.

27. The method of claim 25, wherein the affect is downregulation.

28. A kit comprising at least one oligonucleotide or primer pair that specifically binds to at least one gene as set forth in Tables 2 or 3 or a combination thereof.

29. The kit of claim 28, wherein the oligonucleotide or primer pair is specific for IGFBP3 or fibronectin.
